(19) United States
(12) Patent Application Publication
(10) Pub. No.: US 2018/0100832 A1
(43) Pub. Date: Apr. 12, 2018

(54) HOLDING MEMBER AND ACOUSTIC WAVE APPARATUS

(71) Applicant: CANON KABUSHIKI KAISHA, Tokyo (JP)

(72) Inventor: Hisafumi Ebisawa, Tokyo (JP)

(21) Appl. No.: 15/724,555

(22) Filed: Oct. 4, 2017

(30) Foreign Application Priority Data
Oct. 11, 2016 (JP) 2016-199861

(51) Int. Cl. G01N 29/06; H03H 9/05; G10K 11/36; G01N 29/24; A61B 8/00

(52) U.S. Cl. CPC G01N 29/0681; H03H 9/058; A61B 8/4494; G01N 29/06; G01N 29/24; G10K 11/36

(57) ABSTRACT

A holding member is provided which is capable of, in an acoustic wave apparatus. The holding member includes: a sheet portion configured to separate an upper space for a subject and a lower space for a lower acoustic matching liquid and to store an upper acoustic matching liquid thereon; and a frame portion configured to tauten the sheet portion in a semi-container shape, and a transmission loss by the sheet portion with respect to an acoustic wave is smaller than a transmission loss by the frame portion with respect to the acoustic wave.

### BACKGROUND OF THE INVENTION

### Field of the Invention

The present invention relates to a holding member and an acoustic wave apparatus.

### Description of the Related Art

Research on subject information acquiring apparatuses using an ultrasonic wave (an acoustic wave) to obtain specific information on the inside of a subject such as a breast is underway. An example of such apparatuses is an ultrasonic apparatus which irradiates a subject with an ultrasonic wave and which receives an echo signal reflected by the subject to generate specific information. Another example is a photoacoustic apparatus which irradiates a subject with laser light and which receives an ultrasonic wave (a photoacoustic wave) created by a photoacoustic effect to generate specific information.

An ultrasonic apparatus according to Japanese Patent Application Laid-open No. 2015-167733 includes a water tank into which a breast is suspended and immersed. A probe arranged in a floor portion of the water tank receives an ultrasonic wave emitted from the breast while mechanically moving within a horizontal plane. Based on the ultrasonic wave, three-dimensional image data of the breast is obtained.

Patent Literature 1: Japanese Patent Application Laid-open No. 2015-167733

### SUMMARY OF THE INVENTION

An acoustic wave apparatus according to Japanese Patent Application Laid-open No. 2015-167733 includes a bed provided with an insertion opening for inserting a subject. A rubber sheet portion is installed so as to cover the insertion opening. The subject is immersed together with the sheet portion into a matching liquid stored in a vessel arranged below the sheet portion, and an acoustic wave is detected by a probe array arranged in a bottom portion of the vessel.

The sheet portion reduces a lead time for replacing the matching liquid in the vessel for separating the matching liquid existing along an acoustic propagation path into a subject side and a probe side and ensures an effective operating efficiency of the apparatus. In addition, in the acoustic wave apparatus according to Japanese Patent Application Laid-open No. 2015-167733, a web-like member that is less stretchable than the sheet portion is installed so as to overlap with the sheet portion. This web-like portion serves to hold the subject.

Since the web-like member is created from yarns fine enough to transmit an ultrasonic wave, acoustic transmittance of the web-like member is high. In addition, since acoustic impedance of rubber is similar to that of water, acoustic transmittance of the sheet portion is also high. In this manner, Japanese Patent Application Laid-open No. 2015-167733 describes a holding member that holds a subject in an imaging region while reducing attenuation of acoustic wave propagation.

Due to a mode in which the sheet portion and the web-like member slide relative to each other, the subject holding member described in Japanese Patent Application Laid-open No. 2015-167733 is a mechanism for holding the subject together with the holding member in the matching liquid in conformance to the shape of the subject.

However, an extensive examination performed by the present inventors confirmed that the following problems occur in an acoustic wave apparatus including a holding mechanism made of a laminated composite member such as that described in Japanese Patent Application Laid-open No. 2015-167733.

A first problem is interface reflection and interface attenuation that occur due to an interface existing in the holding member. The interface reflection and the interface attenuation originate from an interface between the sheet portion and the web-like member and cause an artifact to occur in an ultrasonic image obtained from an acoustic wave signal. In addition, a second problem is that, since the sheet portion and the web-like member constituting the holding member are flexible members, a position of the subject together with the holding member fluctuates due to inertial motion of the matching liquid accompanying body motion of an examinee and/or movement of the vessel during an inspection period.

The present invention has been made in consideration of the problems described above. An object of the present invention is to provide, in an acoustic wave apparatus that acquires subject information by receiving an acoustic wave, a holding member capable of reducing transmission loss of a subject derived from the holding member and stably holding the subject during an acoustic wave reception inspection period.

The present invention provides a holding member for holding a subject to be measured in an acoustic wave apparatus the holding member comprising,

a sheet portion configured to store an upper acoustic matching liquid and separate the upper acoustic matching liquid and a lower acoustic matching liquid acoustically coupled with a transducer in an acoustic wave apparatus and

a frame portion configured to tauten the sheet portion in a semi-container shape, and

wherein, in a propagation for the acoustic wave, the sheet portion has a smaller transmission loss than that the frame portion has.

According to the present invention, in an acoustic wave apparatus that acquires subject information by receiving an acoustic wave, a holding member capable of reducing transmission loss of a subject derived from the holding member and stably holding the subject during an acoustic wave reception inspection period can be provided.

Further features of the present invention will become apparent from the following description of exemplary embodiments with reference to the attached drawings.

### BRIEF DESCRIPTION OF THE DRAWINGS

FIGS. 1A and 1B are diagrams each showing a configuration of a subject information acquiring apparatus;

FIGS. 2A to 2C are diagrams each showing a configuration of a holding member according to a first embodiment;

FIGS. 3A and 3B are diagrams each showing a configuration example of a frame portion;

FIGS. 4A and 4B are diagrams each showing a configuration example in which a frame portion has an aperture;

FIG. 5 is a diagram showing an installed state of a holding member;

FIG. 6 is a graph showing a relationship between an allowable deformation amount of a holding member and ultrasonic wave frequency;

FIGS. 7A to 7C are diagrams each showing a configuration of a holding member according to a second embodiment;

FIGS. 8A to 8C are diagrams each showing a configuration of the holding member according to the second embodiment; and

FIGS. 9A to 9C are diagrams each showing a configuration of background art.

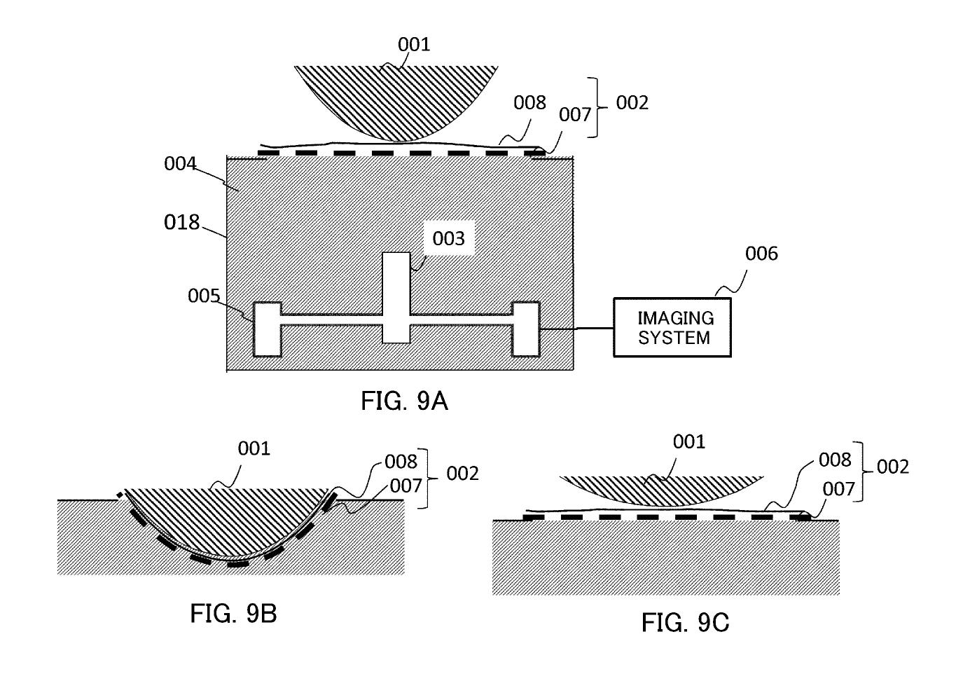

### DESCRIPTION OF THE EMBODIMENTS

Hereinafter, preferred embodiments of the present invention will be described with reference to the drawings. However, it is to be understood that dimensions, materials, shapes, relative arrangements, and the like of components described below are intended to be changed as deemed appropriate in accordance with configurations and various conditions of apparatuses to which the present invention is to be applied. Therefore, the scope of the present invention is not intended to be limited to the embodiments described below.

The present invention relates to a technique for receiving an acoustic wave propagating from a subject and generating and acquiring specific information on the inside of the subject. Accordingly, the present invention can be considered an acoustic wave apparatus or a control method thereof, a subject information acquiring apparatus or a control method thereof, or an acoustic wave receiving method, a subject information acquiring method, or a signal processing method. The present invention can also be considered a program that causes an information processing apparatus including hardware resources such as a CPU and a memory to execute these methods or a storage medium storing the program.

The subject information acquiring apparatus according to the present invention includes an apparatus utilizing a photoacoustic effect in which an acoustic wave generated inside a subject by irradiating the subject with light (an electromagnetic wave) is received and specific information on the subject is acquired as image data. In this case, specific information refers to information on a specific value corresponding to each of a plurality of positions inside the subject which is generated using a received signal obtained by receiving a photoacoustic wave.

Specific information acquired by photoacoustic measurement is a value reflecting an absorption rate of optical energy. For example, specific information includes a generation source of an acoustic wave generated by light irradiation, initial sound pressure inside a subject, optical energy absorption density or an absorption coefficient derived from initial sound pressure, and a concentration of substances constituting tissue. In addition, an oxygen saturation distribution can be calculated by obtaining a concentration of oxygenated hemoglobin and a concentration of reduced hemoglobin as concentrations of substances. Furthermore, a glucose concentration, a collagen concentration, a melanin concentration, a volume fraction of fat or water, and the like are also obtained.

A two-dimensional or three-dimensional specific information distribution is obtained based on specific information at each position in the subject. Distribution data may be generated as image data. Specific information may be obtained as distribution information on respective positions inside the subject instead of as numerical data. In other words, distribution information such as a distribution of initial sound pressure, a distribution of energy absorption density, a distribution of absorption coefficients, and a distribution of oxygen saturation can be obtained.

The subject information acquiring apparatus applied to the present invention includes an ultrasonic apparatus using ultrasonic echo technology which transmits an ultrasonic wave to a subject, receives a reflected wave (an echo wave) that is reflected inside the subject, and acquires subject information as image data. In the case of the ultrasonic apparatus, the acquired subject information is information reflecting a difference in acoustic impedances among tissues inside the subject.

An acoustic wave according to the present invention is typically an ultrasonic wave and includes an elastic wave which is also referred to as a sonic wave or an acoustic wave. An electrical signal transformed from an acoustic wave by a probe or the like is also referred to as an acoustic signal. However, the descriptions of an ultrasonic wave and an acoustic wave in the present specification are not intended to limit a wavelength of the elastic waves. An acoustic wave generated by a photoacoustic effect is referred to as a photoacoustic wave or an optical ultrasonic wave. An electrical signal derived from a photoacoustic wave is also referred to as a photoacoustic signal. In addition, an electrical signal derived from an ultrasonic echo is also referred to as an ultrasound signal.

The subject information acquiring apparatus according to the present invention is capable of being used or is expected to be used in diagnosing vascular diseases and malignant tumors, performing follow-up observations of chemical treatment, and the like with a person or an animal as a subject. In this case, a subject is a part of a living organism. Furthermore, non-living materials such as a phantom can also be measurement subjects.

### First Embodiment

(Apparatus Configuration)

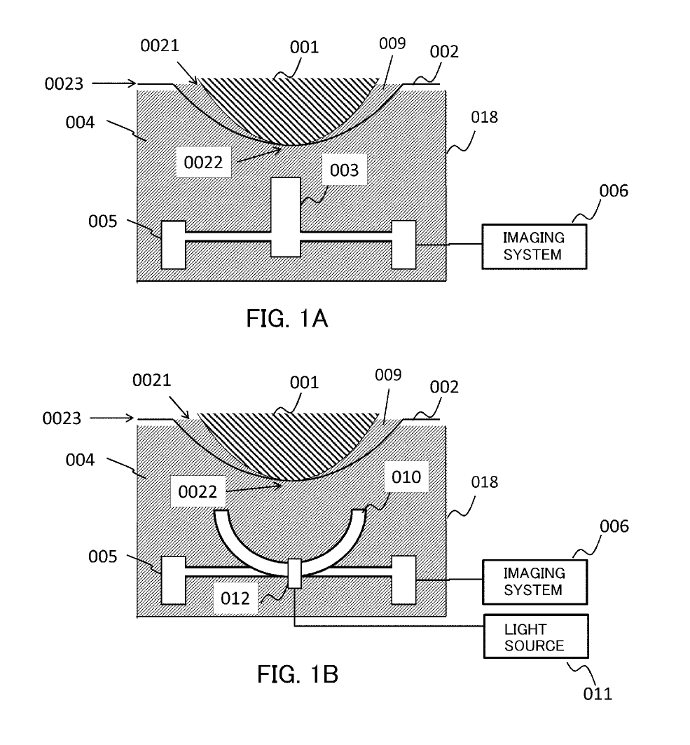

A configuration example in which the present invention is applied to an ultrasonic apparatus will be described with reference to FIG. 1A. Reference numeral 001 denotes a subject (for example, a breast that is a part of a living organism), reference numeral 002 denotes a holding member that holds the subject 001, and reference numeral 003 denotes a probe which transmits an ultrasonic wave and which detects an echo wave from inside the subject 001. An acoustic matching liquid 004 is stored in a vessel 018 configured to allow an acoustic wave propagates from the subject to the probe 003 therethrough. The probe 003 is installed on a driving mechanism 005 (a scanning unit) configured to move inside the acoustic matching liquid 004 in a horizontal direction. Reference numeral 006 denotes an imaging system that converts an echo signal (an ultrasound signal) acquired by the probe 003 into an image.

FIG. 1B represents an example in which the holding member 002 is applied to a photoacoustic apparatus. A light source 011 irradiates the subject 001 with pulsed light through an irradiating unit 012. An element arranged in a bowl-shaped probe 010 receives a sonic wave generated by a photoacoustic effect from hemoglobin in the subject 001 and creates an image of a blood vessel.

(Probe)

The probe 003 in an ultrasonic apparatus transmits an ultrasonic wave (an acoustic wave) to the subject 001. The probe 003 also receives a reflected echo wave propagating from the subject 001 via the holding member 002 and outputs an acoustic wave signal. However, separate probes may be used for transmission and reception. As the probe, a receiving element using a piezoelectric phenomenon, a receiving element using optical resonance, a receiving element using a variation in capacitance, and the like can be used. In addition, using a probe in which a plurality of receiving elements are one-dimensionally or two-dimensionally arranged enables acoustic waves to be simultaneously received at a plurality of locations. As a result, a reduction in measurement time or an expansion of a measurement range can be achieved.

The probe 003 in a photoacoustic apparatus at least has a function for receiving a photoacoustic wave propagating from the subject 001 and converting the photoacoustic wave into an electrical signal. Moreover, depending on the performance of the probe 003, the probe 003 can be used for both photoacoustic measurement and ultrasound echo measurement. In this case, the subject information acquiring apparatus doubles as a photoacoustic apparatus and an ultrasonic apparatus. Moreover, the bowl-shaped probe 010 such as that shown in FIG. 1B can be used in both the photoacoustic apparatus and the ultrasonic apparatus. In the bowl-shaped probe 010, a plurality of receiving elements are arranged so as to form a high sensitivity region in which directions with high receiving sensitivity (directional axes) concentrate. Therefore, high resolution image reconstruction can be performed.

(Light Source)

In the case of a photoacoustic apparatus, the light source irradiates pulsed light with a wavelength which is absorbed by a specific component among components constituting a living organism. As irradiation light, laser light with a pulse width of around 10 to 50 nanoseconds is favorable in order to efficiently generate an acoustic wave. However, a light-emitting diode and a flash lamp may also be used. As the laser, a solid-state laser, a dye laser, a semiconductor laser, and the like are preferable. In addition, when obtaining a concentration of a substance such as oxygen saturation, a wavelength-variable laser may be used. For a living organism, a wavelength of typical pulsed light is 600 to 1100 nm. More typically, the wavelength of the pulsed light is 1 μm or more.

(Irradiating Unit)

When guiding light from the light source to the irradiating unit 012, an optical system such as a bundle fiber, a mirror, a prism, and a photoconductive tube can be used. Moreover, from a safety perspective, a maximum permissible exposure (MPE) is defined as a criterion of an amount of light irradiation with respect to a living organism. The irradiating unit 012 adjusts an irradiation area or an intensity of light so as to satisfy the safety standard.

(Imaging System)

The imaging system 006 performs information processing using an electrical signal (an ultrasound signal derived from a received echo wave or a photoacoustic signal derived from a photoacoustic wave). The imaging system 006 includes a signal processing unit that amplifies and time-sequentially digitizes an electrical signal. The signal processing unit is typically constituted by elements such as a CPU, an amplifier, and an A/D converter and circuits such as an FPGA and an ASIC.

The imaging system 006 also includes an information processing unit that uses a digital signal to perform image reconstruction of the inside of a subject and acquires specific information. As the information processing unit, a PC or a work station that includes computing resources such as a CPU and a memory is preferable. An image reconstruction process is performed in accordance with software programmed in advance. Due to image reconstruction, image data indicating acoustic impedance in the case of an ultrasonic apparatus and image data indicating optical characteristics of a substance in the case of a photoacoustic apparatus are respectively acquired. Any known method such as a phasing addition method and a universal back-projection method can be adopted for image reconstruction.

The imaging system 006 may further include a display unit that displays an image based on image data. As the display unit, a liquid crystal display, an organic EL display, a plasma display, or the like can be used. However, a configuration may be adopted in which image data is stored without providing the display unit. In addition, the imaging system may control the driving mechanism 005 (the scanning unit) to move the probe 003 relative to the holding member 002 to enable imaging over a wider area.

Moreover, favorably, the driving mechanism 005 may also operate the light irradiating unit to implement scanning such that pulsed light avoids a frame portion 007 and is only transmitted through a sheet portion 008. Accordingly, artifacts attributable to the frame portion 007 can be reduced. Alternatively, the imaging system 006 may acquire specific information during image reconstruction by selectively using an acoustic wave generated when the subject is irradiated by pulsed light transmitted through the sheet portion 008.

(Holding Member)

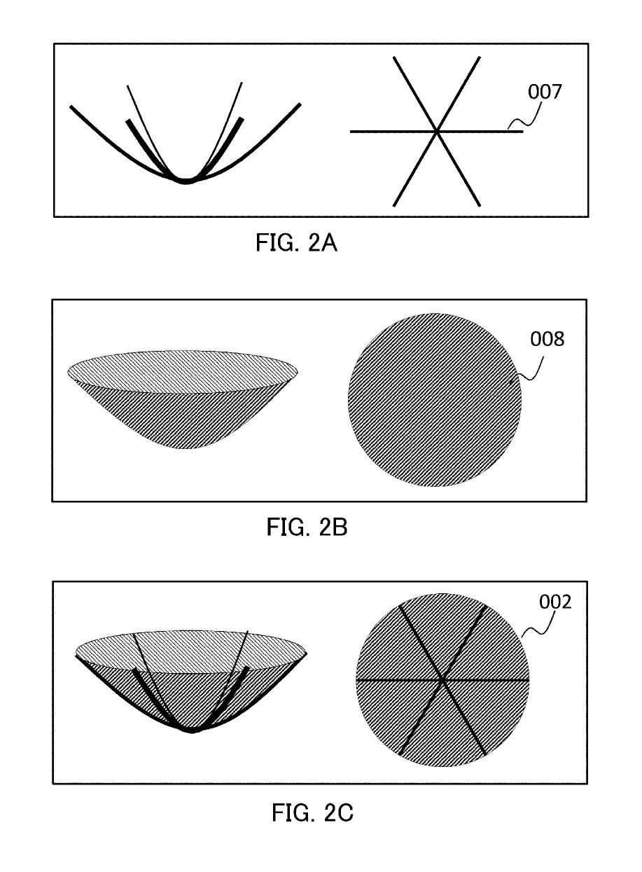

A configuration example of the holding member 002 according to the present invention will now be described with reference to FIGS. 2A to 2D. FIG. 2A is a diagram of the frame portion 007 constituted by a beam-like structure in the holding member 002 as viewed from a side surface and a top surface. FIG. 2B is a diagram of the sheet portion 008 in the holding member 002 as viewed from a side surface and a top surface. FIG. 2C is a diagram of the holding member 002 constituted by the frame portion 007 and the sheet portion 008 described above as viewed from a side surface and a top surface. As illustrated, the holding member 002 is a semi-container shaped member (a semi-container portion) having an aperture for inserting a breast.

The frame portion 007 has a a beam-like structure. The frame portion 007 has an enough strength to maintain its shape under its own weight and even when subjected to external forces from the acoustic matching liquid 004 and the subject 001. The sheet portion 008 is installed along the frame portion 007. The sheet portion 008 is a sheet with high acoustic transmittance. The sheet portion 008 separates an upper space for the subject 001 and a lower space for the acoustic matching liquid 004 in a hygiene manner. In other words, the sheet portion 008 separates the upper acoustic matching liquid 009 and the lower acoustic matching liquid 004. Such a structure enables materials with different components to be used as the acoustic matching liquid 004 on the side of the subject 001 and on the side of the probe 003. A shape of the holding member 002 is determined due to the frame portion 007 having a semi-container shape.

In the present embodiment, water mixed with a surfactant is used on the side of the probe 003 and purified water is used as the acoustic matching liquid 004 on the side of the subject 001. For sanitary reasons, the purified water that comes into contact with the subject 001 is replaced every time imaging is performed. On the other hand, the water on the side of the probe 003 can be repetitively used by performing ultraviolet sterilization. Accordingly, an amount of waste liquid can be reduced while maintaining sanitary conditions. Moreover, a liquid such as oil can be used in addition to water on the side of the probe 003. In addition, besides a liquid such as a skin lotion or oil, a gel or the like with high viscosity can also be used on the side of the subject 001. Furthermore, a pump may be connected to the vessel 018 storing the acoustic matching liquid 004 to enable feeding and discharging. Accordingly, sterilization and liquid amount adjustment can be readily performed.

(Sheet Portion)

Next, the sheet portion 008 will be described. The sheet portion 008 comes into contact with at least a region of interest to be an imaging target in the subject 001 and holds the region. Therefore, acoustic waves emitted from the subject 001 are invariably transmitted through the sheet portion 008 and received by the probe 003. Accordingly, as the sheet portion 008, a material with small acoustic loss (hereinafter, referred to as transmission loss) is desirable. Moreover, transmission loss by the holding member with respect to an acoustic wave will be described as TL. In addition, transmission loss by the sheet portion will be described as TLs (dB) and transmission loss by the frame portion will be described as TLm (dB).

In order to reduce effective transmission loss, favorably, a material with acoustic impedance similar to those of the holding member and the acoustic matching liquid 004 is selected to reduce reflection between the different members. Examples of a material with characteristics similar to those of the acoustic matching liquid 004 include rubbers such as natural rubber, isoprene rubber, and silicone rubber. When the acoustic matching liquid 004 is water, natural rubber or isoprene rubber is favorably used as the holding member. When the acoustic matching liquid 004 is silicone oil, silicone rubber or the like is favorably used as the holding member. In addition, when the acoustic impedance of the sheet portion 008 significantly differs from that of the acoustic matching liquid 004, transmission loss can be reduced by making a thickness of the holding member thinner than a wavelength of the acoustic wave propagating from the subject 001.

Other methods of reducing the transmission loss TL by the holding member include selecting a material with a small acoustic wave attenuation coefficient and relatively reducing a thickness of the sheet. This attenuation coefficient is generally denoted by α (dB/MHz/cm). In addition, transmission loss is defined by the attenuation coefficient α, a frequency of an acoustic wave, and a path length. Therefore, by using a material with a small attenuation coefficient α as the sheet portion 008 and/or using a thin material as the sheet portion 008, the transmission loss TL is further reduced.

In the present embodiment, a PET film with a sheet thickness of 100 μm is used as the sheet portion 008. In this case, the acoustic impedance of the sheet portion 008 is close to 3 and a difference in acoustic impedance from PET and water (with an acoustic impedance of around 1.5) is larger than a difference in acoustic impedance between rubbers and water. However, in the present embodiment, by setting the sheet thickness of the PET film to an extremely thin 100 μm, transmission loss in a thickness direction of the sheet portion 008 can be reduced even when compared to a holding member made of rubbers. Since overall transmission loss by the holding member is determined by material and thickness, by using a holding member made of PET which has higher shape-maintaining performance than a holding member made of rubbers, thickness can be reduced to a level at which a disparity in the difference in acoustic impedance attributable to a difference in materials can be compensated. Moreover, a wavelength and a frequency of an acoustic wave may also be factors when reducing acoustic loss. A representative frequency of the acoustic wave observed in the present invention is 1 MHz.

Conditions related to hydraulic resistance of the sheet portion 008 when using a liquid containing water as a main component as the acoustic matching liquid 004 will be examined. Favorably, the sheet portion 008 is a continuing film having hydraulic resistance of 1000 mmAq or higher as defined by JIS K6404-3: 2015 with respect to the acoustic matching liquid 004. More favorably, the sheet portion 008 is a continuing film having hydraulic resistance of 2000 mmAq or higher as defined by JIS K6404-3: 2015 with respect to the acoustic matching liquid 004. By satisfying such conditions, functions of holding and maintaining the shape of the subject and separating the interior from the exterior of the film can be sufficiently fulfilled.

Moreover, the material of the holding member 002 is not limited to PET and may be another resin-based material or rubber. In addition, when the imaging system 006 is a photoacoustic apparatus, a material with high light transmittance and low light absorptivity is favorable as the sheet portion 008. Favorably, the transmittance of the sheet portion 008 with respect to pulsed light is 50% or higher. For example, transparent isoprene rubber, silicone rubber, urethane rubber or a transparent resin film made of polyethylene terephthalate (PET), polyethylene (PE), or the like is suitable.

(Preferable Configuration Example)

A preferable range will be described in which the transmission loss TL can be reduced while maintaining strength of the holding member 002 using materials such as those described above. It is assumed that a volume density of the sheet portion is denoted by ρs (kg/m³), a thickness in a direction of propagation of a longitudinal wave of the acoustic wave through the sheet portion is denoted by ts (m), a volume density of the frame portion is denoted by ρm (kg/m³), and a thickness in a direction of propagation of the longitudinal wave of the acoustic wave through the frame portion is denoted by tm (m). When these numerical values are given, general expressions (1) and (2) below are favorably satisfied for the transmission loss TLs (dB) by the sheet portion 008 and the transmission loss TLm (dB) by the frame portion 007 with respect to an acoustic wave with a frequency of f (Hz).

TLs=20×log(f×ρs×ts)−43 (1)

TLm=20×log(f×ρm×tm)−43 (2)

In addition, from the perspective of making the transmission loss by the sheet portion 008 smaller than the transmission loss by the frame portion 007, general expression (3) below is favorably satisfied.

TLs<TLm (3)

With respect to a difference in transmission loss in this case, the transmission loss TLs by the sheet portion and the transmission loss TLm by the frame portion favorably satisfy general expression (4) below.

5≦TLm−Tls (dB) (4)

Furthermore, the transmission loss TLs by the sheet portion and the transmission loss TLm by the frame portion favorably satisfy general expression (5) below.

TLm−Tls≦20 (dB) (5)

For example, one-shot molding is performed by adopting PET as the material of the holding member 002. In addition, it is assumed that the frequency f (Hz)=1 MHz. In this case, a general volume density of PET is 1.34 to 1.39 (g/cm³) and, as a representative value, the volume density ρs (kg/m³) of the sheet portion and the volume density ρm (kg/m³) of the frame portion are assumed to be 1.38 (g/cm³).

If the thickness of the sheet portion 008 is assumed to be 80 μm and the thickness of the frame portion 007 is assumed to be 0.5 mm, then TLs≅57.9 (dB) and TLm≅73.8 (dB). In this case, a difference is TLm−TLs=15.9 which satisfies both expressions (4) and (5).

In addition, if the thickness of the sheet portion 008 is assumed to be 100 μm and the thickness of the frame portion 007 is assumed to be 0.75 mm, then TLs≅59.8 (dB) and TLm≅77.3 (dB). In this case, a difference is TLm−TLs=17.5 which satisfies both expressions (4) and (5).

(Frame Portion)

A sheet material for which transmission loss TLs has been reduced by reducing thickness has low rigidity and low holding force. Therefore, a variation in the shape of the subject 001 readily occurs during imaging. This variation causes acquired image data to deteriorate. For example, there may be cases where the imaging system 006 acquires three-dimensional volume data by acquiring image data at respective positions while using the scanning unit to operate the probe 003 to implement scanning in an imaging region and then stacking the image data. When the shape of the subject varies during the scan, a distortion occurs in the volume data. In addition, when reconstructing a group of received signals acquired at respective positions of the probe 003 by delayed processing, since a variation in the value of the subject 001 causes a phase shift in each received signal, resolution deteriorates.

In the present embodiment, in order to suppress a variation (a change in shape) of the subject 001 during imaging, the sheet portion 008 is supported by the frame portion 007. As the frame portion 007, a material that is highly rigid and less likely to vary is favorable. However, since a highly rigid material has higher acoustic impedance than the acoustic matching liquid 004, the transmission loss TL by the holding member increases. In addition, since a highly rigid material tends to have a large sectional area, a path length also tends to be longer and the transmission loss TL increases in proportion to the path length. Therefore, a structure that relatively does not block a sonic wave is required for the frame portion 007. In the example shown in FIG. 2A, the shape of the frame portion 007 has a narrow beam structure that conforms to the shape of a breast. As shown in FIG. 2C, the holding member 002 is formed by conforming the sheet portion 008 to the beam structure.

While the frame portion 007 is thicker than the sheet portion 008, the thickness of the frame portion 007 is favorably reduced as much as possible. For example, a thickness tm (m) in a direction in which a longitudinal wave of an acoustic wave propagates through the frame portion 007 is assumed to be a thickness at which the holding member 002 formed by the frame portion 007 by tautening the sheet portion 008 in a semi-container shape can no longer stand on its own when the thickness of the frame portion 007 is set to ½×tm (m) or less. Accordingly, for example, when the holding member 002 is hooked to an insertion opening of a support table, a situation where the shape of the holding member 002 changes and the holding member 002 sinks into the lower acoustic matching liquid can be prevented.

Moreover, an S/N ratio of a reconstructed image declines as an acoustic wave is blocked by the frame portion 007. In other words, when there are cases where an acoustic wave is blocked and cases where an acoustic wave is not blocked depending on imaging positions, contrast non-uniformity occurs in the reconstructed image and image quality deteriorates. Particularly, in the case of an ultrasonic apparatus, since the existence of a tumor is confirmed based on a difference in contrasts in a reconstructed image, contrast non-uniformity must be reduced as much as possible.

Generally, with an ultrasonic apparatus image for a breast, a contrast resolution of 3 dB is desired in order to diagnose a tumor. When an amount (1−TL) of acoustic waves after reduction due to transmission loss is defined as acoustic transmittance AT, the acoustic transmittance of the sheet portion 008 is denoted by ATs, and the acoustic transmittance of the frame portion 007 is denoted by ATm, expression (6) below is desirably satisfied in order to limit contrast non-uniformity due to the presence or absence of the frame portion 007 to or below 3 dB.

0.7≦ATm/ATs≦1.0 (6)

In addition, when a ratio of acoustic waves transmitted through the frame portion 007 among the acoustic waves used for image reconstruction is R₁, a relationship with acoustic transmittance desirably satisfies expression (7) below.

0.7≦(ATm×R₁+ATs×(1−R₁))/ATs (7)

For example, expression (8) below is desirably satisfied when R₁ is 50%.

0.7≦(ATm×0.5+ATs×(1−0.5))/ATs (8)

In order to have the holding member support a subject in a preferable manner while maintaining its own shape, a material with a higher modulus of rigidity or a higher Young's modulus than the sheet portion 008 is desirable as the material of the frame portion 007. For example, as the frame portion 007, a metal such as iron, SUS (stainless steel), and aluminum or a resin material such as acrylic and polycarbonate can be used. Moreover, rigidity includes shear rigidity and flexural rigidity.

When the imaging system 006 is a photoacoustic apparatus, the frame portion 007 itself generates a photoacoustic wave and may possibly cause an artifact. In consideration thereof, as the frame portion 007, a material that is less likely to absorb light is favorable. Materials that are less likely to absorb light include a transparent material such as acrylic and polycarbonate and a material with a surface subjected to a metal coating process in order to increase reflectance of light. In the present embodiment, the frame portion 007 in which a gold coating process is applied to a surface of SUS is used. Moreover, besides gold, using a metal with high reflectance in the infrared region such as silver and aluminum is also effective.

(Shape of Frame Portion)

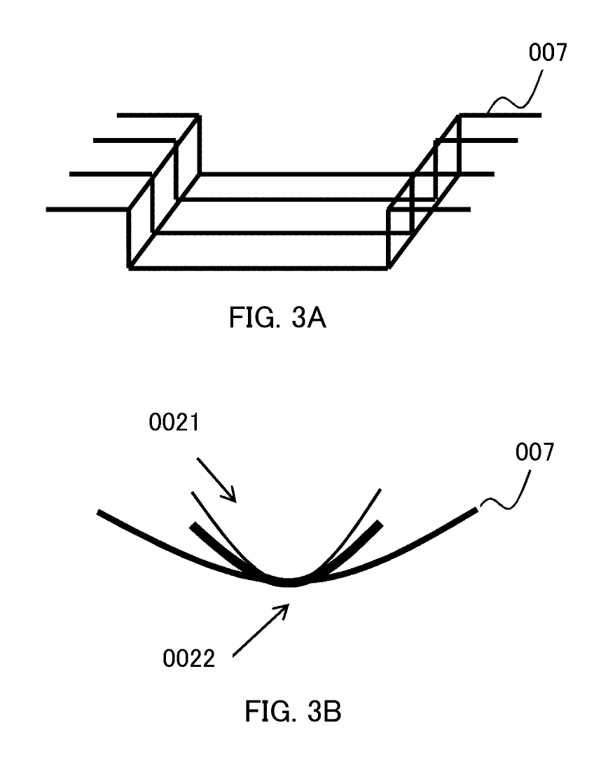

When placing the subject 001 in the holding member 002, significantly compressing or deforming the shape of the subject 001 poses a burden on an examinee. Therefore, the used holding member 002 is favorably shaped so as to conform to the shape of the subject 001. Since a large number of parts of living organisms including a breast are rounded, the frame portion 007 favorably also has a rounded shape. However, when the subject 001 has a relatively nearly flat shape such as the palm of a hand or a sole of a foot, a flat frame portion 007 may be used as shown in FIG. 3A.

In the present embodiment, a cup-shaped frame portion 007 with a rounded protruded portion is used. As a result, the holding member 002 can be described as having a protruding shape that protrudes toward a side of a probe (not shown) or having a depressed shape toward a subject (not shown). Specifically, the holding member 002 has an aperture 0021 and an apex 0022 at a distance from the aperture 0021, and a subject is inserted on a side of the depressed portion of the holding member 002 from the aperture 0021. For example, when an acoustic wave apparatus has a support table which supports an examinee and which is provided with an insertion opening into which a subject is inserted, the subject is held by the holding member 002 arranged below the insertion opening.

Therefore, when connecting the holding member to the support table, the holding member is connected so that the aperture of the holding member is at a position overlapping with the insertion opening of the support table in a vertical direction. In addition, in the present embodiment, since the examinee lies face-down on the support table, a positional relationship is adopted in which the apex 0022 of the holding member is lower than an imaginary plane 0023 in the vertical direction.

When observing a deep part of a highly flexible subject 001 such as a breast, in order to reduce transmission loss in the subject 001, there is a method of compressing the subject 001 to reduce a distance between the probe and the observation portion. Even in a case where the shape of the subject 001 is varied as described above, a burden on an examinee is desirably reduced as much as possible. Therefore, the shape of the subject 001 after compression is favorably similar to the original shape of the subject 0001. To this end, as shown in FIG. 3B, using a frame portion 007 with a smaller curvature than in FIGS. 2A to 2D is effective. In addition, as shown in FIG. 3A, it is also effective to use the frame portion 007 of which an installation surface is flat but which also includes a depression for housing a volume corresponding to a breast.

(Improving Holding Force)

Applying a coupling process to the frame portion 007 and the sheet portion 008 is effective in increasing a holding force of the holding member 002 as a whole. When using a resin film or the like with low stretchability as the sheet portion 008, variation in a stretching direction can be reduced by performing a coupling process with the frame portion 007. In addition, when the sheet portion 008 is a plate material (for example, a resin plate or a metal plate) with relatively high rigidity, variation in a torsional direction of the sheet portion 008 can be reduced by coupling the sheet portion 008 to the frame portion 007. By having the frame portion 007 tauten the sheet portion 008 and then gluing the two portions together, a tautened state can be more readily maintained while supporting the subject 001 and the acoustic matching liquid 004.

Methods of the coupling process include using an adhesive and ultrasonic bonding. In the present embodiment, a transparent and water-resistant adhesive is used. As another method, when molding the sheet portion 008, the frame portion 007 and the sheet portion 008 are coupled by placing preform pellets of the frame portion 007 and the sheet portion 008 together in a molding die and performing molding. As yet another method, the frame portion 007 is placed in a rubber liquid and extracted therefrom to form a film by surface tension which is subsequently hardened.

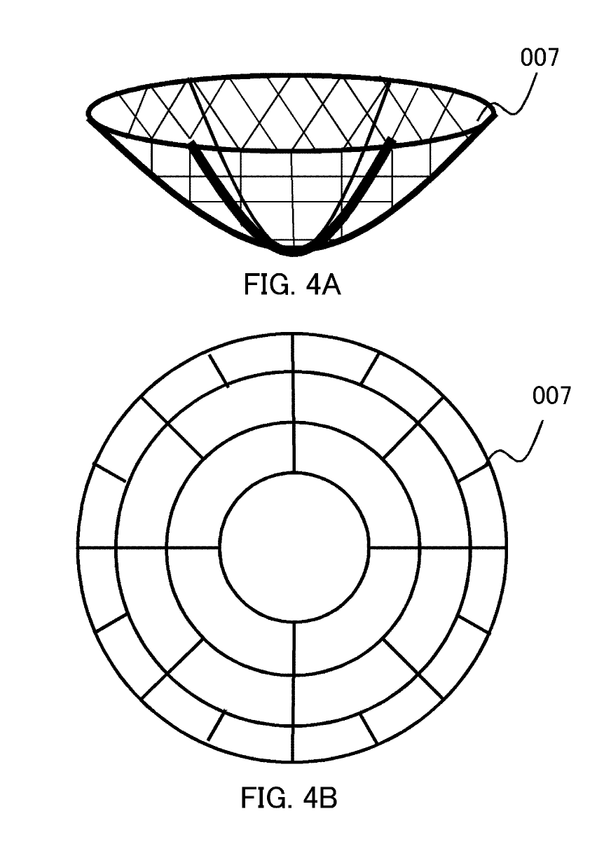

Vertically and horizontally arranging beams as shown in FIG. 4A is also effective in increasing holding force. Configurations of the beams include a radial shape, a honeycomb shape, and a truss structure. However, when an area occupied by beam portions in the holding member 002 becomes larger, although holding force increases, transmittance of an acoustic wave declines. Therefore, beams are favorably reduced in portions in contact with a region near an imaging location (a region of interest) in the subject 001 in order to increase an aperture ratio. For example, when the subject 001 is a breast, a vicinity of a center of the holding member 002 often becomes an imaging location (a region of interest). In consideration thereof, as shown in FIG. 4B, an aperture ratio of a central portion of the holding member 002 is favorably set higher than an aperture ratio of peripheral portions.

Depending on a shape of the frame portion 007, an S/N ratio of a reconstructed image declines due to the frame portion 007 blocking an acoustic wave. In other words, when there are cases where an acoustic wave is blocked and cases where an acoustic wave is not blocked depending on imaging positions, contrast non-uniformity occurs in the reconstructed image and image quality deteriorates. As described earlier, generally, with an ultrasonic apparatus image for a breast, contrast non-uniformity is favorably limited to or below 3 dB. In order to limit the contrast non-uniformity due to the frame portion 007 to or below 3 dB, an aperture ratio (a proportion of a sheet portion region in the entire holding member 002) is desirably set to 70% or higher.

(Maintaining Shape)

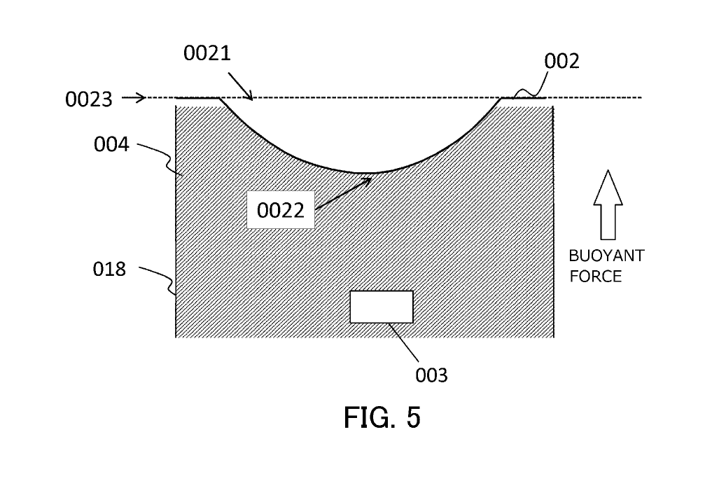

FIG. 5 shows a situation where the lower acoustic matching liquid 004 is filled in the vessel 018 under the holding member 002 and No upper matching liquid is stored in the holding member 002 thereon. In FIG. 5, An upper storing space in the holding member 002 is empty. In this case, the acoustic matching liquid 004 stored in the vessel 018 pushed upwardly the holding member 002. As a result, the holding member 002 receives a buoyant force in accordance with a volume of the acoustic matching liquid 004 pushed aside and is pushed upwardly to an upper side in the vertical direction. At this point, when strength of the holding member 002 is insufficient, the holding member 002 is squashed by the buoyant force, making it difficult to insert the subject 001. In addition, since the acoustic matching liquid 004 on the side of the subject 001 cannot readily penetrate a squashed portion, bubbles remain on the surface of the subject 001 when placing the subject 001. Bubbles have low acoustic wave transmittivity and may cause artifacts.

Therefore, the holding member 002 is made strong enough to maintain a shape conforming to the subject 001 even in a state where the side of the probe 003 is filled with the acoustic matching liquid 004. In other words, the holding member 002 is created such that, even when the holding member 002 secured to the support table receives a buoyant force, a portion immersed in the acoustic matching liquid 004 retains a curved shape protruding downwardly.

In addition, image quality of a reconstructed image improves when the shape of the holding member 002 can be maintained in a state where the subject 001 is placed on the holding member 002. This is because acquiring a distance between the acoustic matching liquid 004 and the subject 001 on a path of an acoustic wave based on the shape of the holding member 002 enables reconstruction in consideration of a sound velocity of each region to be readily performed.

A case where a positional relationship between the imaginary plane 0023 including the aperture of the holding member 002 and the apex is defined will now be considered. An ability of the holding member 002 to retain its shape may be considered an ability of maintaining a polarity of a curvature of the holding member 002 when the holding member 002 is reversed from a state where the apex 0022 is below the aperture 0021 in the vertical direction to a state where the apex 0022 is above the aperture 0021 in the vertical direction.

(Allowable Deformation Amount)

Moreover, it may be assumed that the holding member 002 deforms to a certain degree. For example, when the subject 001 is a breast, a load applied to the holding member 002 can be assumed to be 30 N toward a protruding side of the frame portion 007. In addition, a contact region of the subject 001 and the holding member 002 can be assumed to be a circular region with a diameter of 50 mm. The holding member 002 is required to be strong enough to reduce an amount of shape deformation under such assumptions to within an allowable range. However, the numerical values described above are merely an example. Since an assumed load varies depending on a size of a breast, numerical values are not limited to the above.

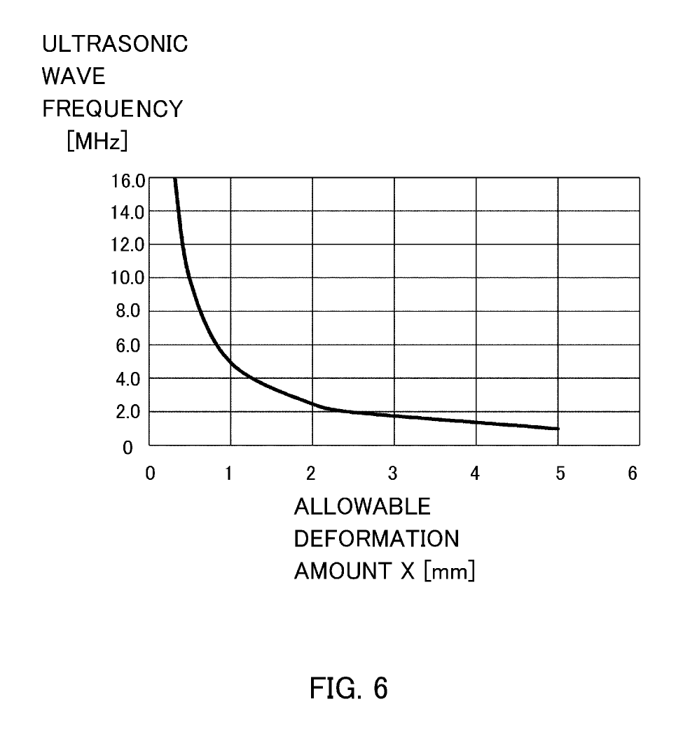

FIG. 6 is a graph showing an allowable deformation amount in accordance with an ultrasonic wave frequency of the holding member 002 in the example described above. The allowable deformation amount is calculated from a sound velocity of the respective acoustic matching liquids 004 on the side of the subject 001 and the side of the probe 003, a sound velocity of the subject 001, and a frequency of an ultrasonic wave. An amount of deformation of the holding member falls within a prescribed range such as that represented by the graph between a first state in which the subject 001 is supported and the acoustic matching liquid 004 is held and a second state in which the subject 001 is not supported and the acoustic matching liquid 004 is not held.

Generally, a phase shift error of a group of received signals used to create a reconstructed image is required to be limited to or below 1/16 of a wavelength of a received signal in the case of a high image quality mode. In addition, even in the case of a general image quality mode, the phase shift error is required to be limited to or below ¼ of a wavelength of a received signal. When assuming that the acoustic matching liquid 004 on the side of the probe 003 is purified water at 20° C. to 40° C. and the subject 001 is a breast, respective sound velocities are assumed to be 1480 to 1530 m/s and 1420 to 1530 m/s. If the holding member 002 varies by X m from an assumed position, an assumed maximum phase shift amount of a received signal is calculated as follows from a deformation amount X and the sound velocities described above.

|X/1530−X/1420| (seconds)

FIG. 6 shows a frequency at which a wavelength with respect to the allowable deformation amount X is ¼. FIG. 6 indicates that, with an ultrasonic apparatus having a main frequency of 10 MHz, a deformation amount X before and after holding is desirably limited to within ±0.5 mm. In addition, it is shown that with a photoacoustic apparatus having a main frequency of 2 MHz, a deformation amount is desirably limited to within ±2.5 mm. Generally, with a photoacoustic apparatus, there is a tendency that a low shape maintaining capability of the holding member 002 is more likely to be allowed as compared to an ultrasonic apparatus. As a result, a wider range of materials and structures can be used for the frame portion 007.

(Positional Relationship between Frame Portion and Sheet Portion)

A positional relationship between the frame portion 007 and the sheet portion 008 in the holding member 002 will be described. When the sheet portion 008 is made of a relatively hard material such as a resin, the sheet portion 008 may be arranged on a side in contact with the subject 001. In this case, the sheet portion 008 is to be positioned on an inner side of the frame portion 007 in a depressed portion formed by the holding member 002. Accordingly, a load surface to the subject 001 becomes wider and a load applied to the subject 001 decreases. In addition, when the sheet portion 008 is made of a soft material such as rubber, the sheet portion 008 becomes a buffer. As a result, a load applied to the subject 001 can be reduced and effects such as improved comfortability of the examinee and preventing the frame portion 007 from leaving a mark on the subject 001 can be produced.

Moreover, when the sheet portion 008 exists on the side of the subject 001 as described above, the frame portion 007 and the sheet portion 008 are desirably integrally coupled with each other. Accordingly, a deformation of the sheet portion 008 due to a buoyant force from the acoustic matching liquid 004 can be prevented. As a result, effects such as making installation of the acoustic matching liquid 004 on the side of the subject 001 and insertion of the subject 001 easier and preventing introduction of bubbles are produced.

On the other hand, arranging the frame portion 007 on the side of the subject 001 enables a deformation of the sheet portion 008 due to a buoyant force from the acoustic matching liquid 004 to be prevented. When the frame portion 007 is arranged on the side of the subject 001 in this manner, in order to reduce a load applied to the subject 001, a contact surface of the frame portion 007 with the subject 001 is favorably subjected to a process such as a planarization process, a smoothing process, or a buffer application process.

Using the holding member 002 described above in a subject information acquiring apparatus in which the acoustic matching liquid 004 exists between the subject 001 and the probe 003 enables the acoustic matching liquid 004 to be readily installed before placing the subject 001. In addition, the subject 001 can be placed at a prescribed position. As a result, the holding member 002 capable of reducing acoustic loss and capable of fixing the subject 001 during imaging can be realized.

### Second Embodiment

In the present embodiment, a configuration of the holding member 002 which differs from the first embodiment will be described. Moreover, a subject information acquiring apparatus to which the holding member 002 is applied is the same as that according to the first embodiment.

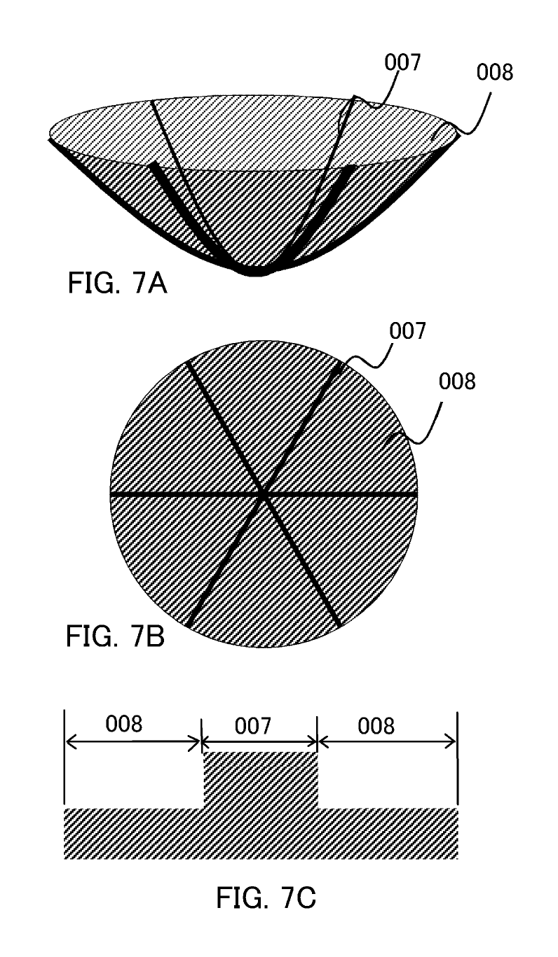

In the present embodiment, the sheet portion 008 and the frame portion 007 are constructed of a same material. In addition, the holding member 002 including both portions is molded in one shot. FIGS. 7A to 7C show an example of a mode of the holding member 002. The material of the holding member 002 is PET and the holding member 002 is created by injection molding. As shown in the sectional view in FIG. 7C, the frame portion 007 and the sheet portion 008 have different thicknesses with the frame portion 007 being thicker. This is because while the sheet portion 008 is favorably made thinner in order to reduce transmission loss TL, the frame portion is favorably made thicker in order to increase rigidity. As a yardstick, the thickness of the sheet portion 008 is favorably shorter than a wavelength of an acoustic wave used for imaging. On the other hand, the thickness of the frame portion 007 may be set longer than the wavelength of the acoustic wave in order to ensure the holding force described in the first embodiment.

Since a coupling force of the sheet portion 008 and the frame portion 007 increases by molding both portions together, an increase in the holding force can be expected. In addition, since an adhesive is not used, an acoustic signal originating from an adhesive is not created. As a result, an improvement in image quality can be expected.

(Modifications)

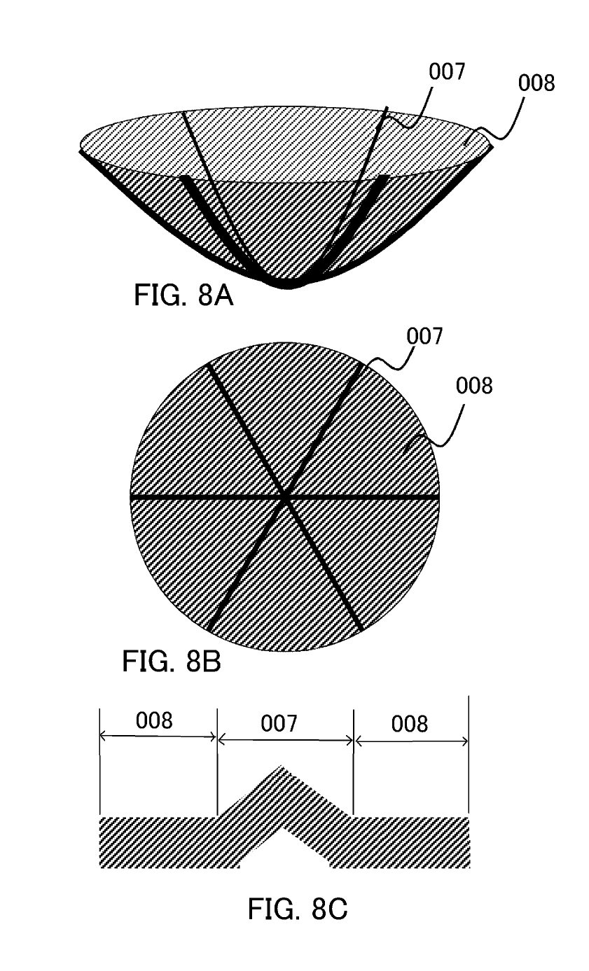

Next, an example of increasing the holding force of the holding member 002 by modifying a shape thereof instead of varying the thicknesses of the sheet portion 008 and the frame portion 007 will be described. The holding member 002 shown in FIGS. 8A to 8C is created by pneumatically molding a PET sheet. As indicated by a section shape shown in FIG. 8C, a bent portion is provided in the frame portion 007 of the holding member 002. Due to the frame portion 007 having a bent portion that reduces deformation when pressure is applied in a direction from an outer side toward an inner side of a depressed portion formed by the holding member 002, strength is improved.

Moreover, in the present embodiment, the material of the holding member 002 is not limited to PET. However, in the case of a photoacoustic apparatus, a transparent resin material that does not absorb light is desirable. In addition, a molding method other than the methods described in the present embodiment may be used. The shape and strength required by the material of the holding member 002 are the same as those described in the first embodiment and a shape other than those shown in FIGS. 7A to 7C and 8A to 8C can be adopted.

In FIGS. 7A to 7C and 8A to 8C, the protrusion of the frame portion 007 is favorably shaped so as to protrude on the side of the probe 003. Accordingly, a load applied to the subject 001 can be reduced. In this case, as shown in FIGS. 7A to 7C and 8A to 8C, each beam of the frame portion 007 is favorably formed so that a longitudinal direction thereof is aligned with an up-down direction during use of the holding member 002. Accordingly, bubbles adhering on the side of the probe 003 of the holding member 002 flow upward and are less likely to remain adhered.

Using the holding member 002 described above enables the acoustic matching liquid 004 to be installed before placing the subject 001, enables the subject 001 to be placed at a prescribed position, and enables the holding member 002 capable of reducing acoustic loss and capable of fixing the subject 001 during imaging to be created in one shot.

The present invention can also be implemented by a computer (or a device such as a CPU or an MPU) of a system or an apparatus that realizes the functions of the embodiments described above by reading and executing a program recorded on a storage apparatus. In addition, the present invention can also be implemented by a method constituting steps to be executed by computer of a system or an apparatus that realizes the functions of the embodiments described above by reading and executing a program recorded on a storage apparatus. Alternatively, the present invention can also be achieved by a circuit (for example, an ASIC) that realizes one or more functions. To this end, for example, the computer is provided with the program via a network or from various types of recording media capable of functioning as the storage apparatus (in other words, a computer-readable recording medium that stores data in a non-transitory manner). Therefore, all of the computer (including a device such as a CPU or an MPU), the method, the program (including a program code or a program product), and the computer-readable recording medium that stores the program in a non-transitory manner described above fall within the scope of the present invention.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-199861, filed on Oct. 11, 2016, which is hereby incorporated by reference herein in its entirety.

What is claimed is:

1. A holding member for holding a subject to be measured in an acoustic wave apparatus, the holding member comprising:
a sheet portion configured to store an upper acoustic matching liquid and separate the upper acoustic matching liquid and a lower acoustic matching liquid acoustically coupled with a transducer in an acoustic wave apparatus and
a frame portion configured to tauten the sheet portion in a semi-container shape, and
wherein, in a propagation for the acoustic wave, the sheet portion has a smaller transmission loss than that the frame portion has.

2. The holding member according to claim 1, wherein
a transmission loss TLs (dB) by the sheet portion and a transmission loss TLm (dB) by the frame portion with respect to an acoustic wave with a frequency of f (Hz) satisfy general expressions (1), (2), and (3) below when a volume density of the sheet portion is ρs (kg/m³), a thickness in a direction of propagation of a longitudinal wave of the acoustic wave through the sheet portion is ts (m), a volume density of the frame portion is ρm (kg/m³), and a thickness in a direction of propagation of the longitudinal wave of the acoustic wave through the frame portion is tm (m):
TLs=20×log(f×ρs×ts)−43 (1)
TLm=20×log(f×ρm×tm)−43 (2)
TLs<TLm (3).

3. The holding member according to claim 1, wherein
with respect to an acoustic wave with a frequency of 1M (Hz), the transmission loss by the sheet portion is smaller than the transmission loss by the frame portion.

4. The holding member according to claim 2, wherein
the transmission loss TLs by the sheet portion and the transmission loss TLm by the frame portion satisfy general expression (4) below:
5≦TLm−Tls (dB) (4).

5. The holding member according to claim 4, wherein
the transmission loss TLs by the sheet portion and the transmission loss TLm by the frame portion satisfy general expression (5) below:
TLm−Tls≦20 (dB) (5).

6. The holding member according to claim 1, wherein
a modulus of rigidity of the frame portion is higher than a modulus of rigidity of the sheet portion.

7. The holding member according to claim 1, wherein
the holding member includes an aperture communicating with the sheet portion having a semi-container shape from the outside and an apex at a distance from an imaginary plane including the aperture, and
a polarity of a curvature of the holding member is maintained when the holding member is reversed from a state where the apex is below the imaginary plane in a vertical direction to a state where the apex is above the imaginary plane in the vertical direction.

8. The holding member according to claim 7, wherein
a thickness tm (m) in a direction in which a longitudinal wave of the acoustic wave propagates through the frame portion is a thickness at which the holding member formed by the frame portion by tautening the sheet portion can no longer stand on its own when the thickness of the frame portion is set to ½×tm (m) or less.

9. The holding member according to claim 1, wherein
the sheet portion is provided on a side in contact with the subject in the holding member.

10. The holding member according to claim 1, wherein
the frame portion and the sheet portion are integrally coupled.

11. The holding member according to claim 10, wherein
the frame portion and the sheet portion are constructed of a same material.

12. The holding member according to claim 6, wherein
the frame portion includes a material with a higher Young's modulus than the sheet portion.

13. The holding member according to claim 6, wherein
a thickness of the frame portion is greater than a thickness of the sheet portion.

14. The holding member according to claim 6, wherein
the frame portion has a bent portion that reduces deformation of the holding member when pressure is applied in a direction from an outer side toward an inner side of a depressed portion formed by the holding member.

15. An acoustic wave apparatus, comprising:
the holding member according to claim 1; and
a support table configured to support an examinee and having an insertion opening through which the subject is inserted, which is a part of the examinee, is arranged, wherein
the holding member is connected to the support table at a position overlapping with the insertion opening,
the acoustic wave apparatus further comprising:
a probe that receives an acoustic wave propagated from the subject via the holding member and that outputs an acoustic wave signal; and
a vessel that stores the lower acoustic matching liquid between the probe and the holding member; and
wherein even when the holding member receives a buoyant force based on the lower acoustic matching liquid stored in the vessel, a portion of the holding member which is immersed in the lower acoustic matching liquid is secured to the support table and still has a curved shape protruding downwardly.

16. The acoustic wave apparatus according to claim 15, further comprising
a light irradiating unit that irradiates the subject with pulsed light via the holding member.

17. The acoustic wave apparatus according to claim 16, wherein
the sheet portion has a transmittance of 50% or higher with respect to the pulsed light.

18. The acoustic wave apparatus according to claim 16, wherein
a wavelength of the pulsed light is 1 μm or longer.

19. The acoustic wave receiving apparatus according to claim 15,
further comprising a scanning unit configure to move the probe relative to the holding member.

20. The acoustic wave apparatus according to claim 15,
further comprising an information processing unit that acquires specific information on the subject based on the acoustic wave signal
wherein
the information processing unit acquires the specific information on the subject based on the acoustic wave generated when the subject is irradiated with the pulsed light having transmitted through the sheet portion.

21. The acoustic wave apparatus according to claim 15, wherein
at least any one of the upper acoustic matching liquid and the lower acoustic matching liquid contains water as a main component, and
the sheet portion is a continuing film having hydraulic resistance of 1000 mmAq or higher as defined by JIS K6404-3: 2015.
